(19) United States
(12) Patent Application Publication
(10) Pub. No.: US 2009/0124977 A1
(43) Pub. Date: May 14, 2009

(54) Method and Apparatus for Reversing a Piston Rod in an Injection Device

(75) Inventor: Bo Vestergard Jensen, Copenhagen V (DK)

Correspondence Address: NOVO NORDISK, INC.;INTELLECTUAL PROPERTY DEPARTMENT, 100 COLLEGE ROAD WEST, PRINCETON, NJ, 08540, US

(73) Assignee: Novo Nordisk A/S, Bagsvaerd (DK)

(21) Appl. No.: 11/816,550

(22) PCT Filed: Jan. 31, 2006
(86) PCT No.: PCT/DK06/00049

§ 371 Date: Oct. 16, 2007

(30) Foreign Application Priority Data
Feb. 23, 2005 (DK) PA 2005 00275

(51) Int. Cl. A61M 5/145

(52) U.S. Cl. 604/154

(57) ABSTRACT

The present invention relates to a method for moving a piston rod in an injection device, the piston rod being operatively connected to a motor shaft of an electrical motor so that a rotational movement of the motor shaft is transformed to a translational movement of the piston rod, the piston rod being adapted to be moved between two end positions, the method comprising the steps of providing a first supply voltage level to the electrical motor, measuring sets of corresponding values of current provided to the motor and a corresponding position of the piston rod, and providing a second supply voltage level to the electrical motor when the measured position of the piston rod equals predetermined positions, the second supply voltage level having a RMS value being smaller than the RMS value of the first supply voltage level. The present invention further relates to an apparatus for carrying out the method.

### FIELD OF THE INVENTION

The present invention relates to a method and an apparatus for reversing a piston rod in an injection device. In particular, the present invention relates to a method for reversing a piston rod in an injection device using information about current provided to a DC-motor operatively connected to a piston rod, and the position of the piston rod.

### BACKGROUND OF THE INVENTION

Injection devices with DC-motors are well known in the field of dispensing devices. In these devices a piston rod is driven by an electrical DC-motor. When the piston rod has reached its outermost position and the medicine containing reservoir containing the medicament to be injected is empty the piston rod needs to be reversed to its initial position so that the medicine containing reservoir can be replaced.

US 2004/0085215 discloses a pump, a reservoir and a reservoir piston for providing controlled delivery of fluids. A motor is operably coupled to a drive member, such as a drive screw, which is adapted to advance a plunger slide in response to operation of the motor. The plunger slide is removably coupled to the piston. The electrical current to an infusion pump is measured. Based on the current measurements, the infusion pump detects when the plunger slide is seated in the reservoir, and detects a problem with a force sensor when the force sensor independently fails to register a value indicating that the plunger slide is seated in the reservoir.

US 2004/0085215 is silent about how the piston is reversed to its initial position after it has reached its outermost position and the medicine containing reservoir is empty.

It is an object of the present invention to provide a method and an apparatus for providing a safe return of a piston rod in an injection device.

### SUMMARY OF THE INVENTION

The above-mentioned object is complied with by providing, in a first aspect, a method for reversing a piston rod in an injection device, the piston rod being operatively connected to a motor shaft of an electrical motor so that a rotational movement of the motor shaft is transformed to a translational movement of the piston rod, the piston rod being adapted to be moved between two end positions, the method comprising the steps of
providing a first supply voltage level to the electrical motor,
measuring sets of corresponding values of current provided to the motor and piston rod position,
shutting down the electrical motor in case the measured current provided to the motor exceeds a predetermined value, and
comparing a measured final position of the piston rod with a predetermined position range.

The electrical motor may be a DC-motor being driven by a motor controller controlled by a microprocessor. The motor controller may comprise an H-bridge transistor configuration. When using an H-bridge it is possible to reverse the motor direction and control the voltage applied to the motor by Pulse Width Modulating (PWM) the H-bridge transistors. The PWM is controlled by the microprocessor system.

The term final position is primarily to be understood as the position of the piston rod after shut down of the electrical motor. However, final position may also be understood as a position of the piston rod during shut down. Thus, the electrical motor may not necessarily be brought to a complete stop before the position of the piston rod is measured.

The position of the piston rod may be measured by measuring a corresponding angular position of the motor shaft or by measuring the actual position of the piston rod.

The predetermined current level is a current level exceeding the current level of a motor operating under normal loading conditions (nominal current level). The predetermined current may in principle be set arbitrary. However, the predetermined current level must be below the current threshold current level where damages due to over currents start to occur—either on the motor controller, the mechanical drive mechanism or in the motor itself. Typically, the predetermined current level falls within the range corresponding to 1.5 and 4 times the nominal current level of the motor.

The predetermined position range may be an interval of positions around an initial position or starting position of the piston rod. The initial position or the starting position is stored in a memory unit. This position is not necessarily a fixed position in that the initial position or starting position is defined as end position of the reversing sequence the last time the piston rod was asked to reverse to its initial position.

The method according to the present invention may further comprise the step of generating an error signal if the measured final position of the piston rod is not included within the predetermined position range. Thus, the case the piston rod is prevented from reversing to its initial position or a position around its initial position an alarm signal is generated telling the user of the injection device that something is wrong. For example it could be that the piston rod meets a mechanical blockage which prevents that the piston rod can return to its initial position.

If the measured final position is included within the predetermined position range the method according to the present invention may further comprise the step of generating an approving signal. The approving signal informs the user of the injection device that the piston rod has returned to its starting position or a position around it, and the empty medicine containing reservoir can now be replaced. The method according to the present invention may further comprise the step of storing the measured final position of the piston rod. The position of the piston rod where the motor was shut down may be stored in an associated memory module as a new starting position. This new starting position may replace an older starting position. This imply, that the next time the piston rod is to be reversed to its starting position the new starting position is loaded into the system, and used as the position to return to.

In a second aspect, the present invention relates to a method for reversing a piston rod in an injection device, the piston rod being operatively connected to a motor shaft of an electrical motor so that a rotational movement of the motor shaft is transformed to a translational movement of the piston rod, the piston rod being adapted to be moved between two end positions, the method comprising the steps of
providing a first supply voltage level to the electrical motor,
measuring sets of corresponding values of current provided to the motor and piston rod position, and
providing a second supply voltage level to the electrical motor in case the measured position of the piston rod equals predetermined positions, the second supply voltage level having a RMS value being smaller than the RMS value of the first supply voltage level.

Again, the position of the piston rod may be measured by measuring a corresponding angular position of the motor shaft.

The predetermined positions may be an interval of positions near the starting position of the piston rod. Thus, when the piston rod approaches its starting position the speed with which the piston rod is returned is slowed down in order to secure a soft landing of the piston rod when it reaches the starting position. By slowing down the speed of the piston rod the precision of the reversing process is increased. Also, the mechanical parts, including the piston rod itself, are also protected against overload and unnecessary wear when the speed is reduced.

The method according the second aspect may further comprise the step of shutting down the electrical motor in case the current provided to the motor exceeds a predetermined value, such as between 1.5 and 4 times the nominal current level of the motor. The method may further comprise the step of comparing a measured final position of the piston rod with a predetermined position range and generating an error signal if the measured final position is not included within the predetermined position range.

On the contrary, if the measured final position is included within the predetermined position range the method may further comprise the step of generating an approving signal and storing the measured final position of the piston rod in an associated memory module for later use.

In a third aspect, the present invention relates to a method for reversing a piston rod in an injection device, the piston rod being operatively connected to a motor shaft of an electrical motor so that a rotational movement of the motor shaft is transformed to a translational movement of the piston rod, the piston rod being adapted to be moved between two end positions, the method comprising the steps of
providing a supply voltage level to the electrical motor,
measuring sets of corresponding values of current provided to the motor and piston rod position, and
changing the supply voltage level if the measured position of the piston rod equals predetermined positions, or if the current provided to the motor exceeds a predetermined value.

In a fourth aspect, the present invention relates to an apparatus for carrying out the method according to the first aspect of the present invention, the apparatus comprising
means for providing a first supply voltage level to the electrical motor,
means for measuring sets of corresponding values of current provided to the motor and piston rod position,
means for shutting down the electrical motor in case the measured current provided to the motor exceeds a predetermined value, and
means for comparing a measured final position of the piston rod with a predetermined position range.

In a fifth aspect, the present invention relates to an apparatus for carrying out the method according to the second aspect of the present invention, the apparatus comprising
means for providing a first supply voltage level to the electrical motor,
means for measuring sets of corresponding values of current provided to the motor and piston rod position, and
means for providing a second supply voltage level to the electrical motor if the measured position of the piston rod equals predetermined positions, the second supply voltage level having a RMS value being smaller than the RMS value of the first supply voltage level.

In a sixth aspect, the present invention relates to an apparatus for carrying out the method according to the third aspect of the present invention, the apparatus comprising
means for providing a supply voltage level to the electrical motor,
means for measuring sets of corresponding values of current provided to the motor and piston rod position, and
means for changing the supply voltage level if the measured position of the piston rod equals predetermined positions, or if the current provided to the motor exceeds a predetermined value.

The means for providing a supply voltage to the electrical motor may be a H-bridge comprising four transistors—two transistors cooperating to drive the motor in a forward direction and two other transistors for driving the motor in a reverse direction. The motor itself may be a DC-motor. The means for measuring the current provided to the motor may be a current transformer or it may be an, in relation to the motor, external resistor over which the voltage drop is measured. The measured representative of the current provided to the motor is converted into a digital format by use of an A/D converter. The position of the piston may be indirectly measured by measuring the angular position of the motor shaft of the motor. For example, a direction sensitive encoder may be used for measuring the angular position of the motor shaft.

### BRIEF DESCRIPTION OF THE DRAWINGS

The present invention will now be described in further details with reference to the accompanying figures, wherein

FIG. 1 shows the system including motor, piston rod control unit etc,

FIG. 2 shows a flow chart illustrating a position calibration procedure,

FIG. 3 shows a flow chart illustrating how the piston rod is reversed to its starting position, and

FIG. 4 shows a measurement of motor current vs. piston rod position.

While the invention is susceptible to various modifications and alternative forms, specific embodiments have been shown by way of example in the drawings and will be described in detail herein. It should be understood, however, that the invention is not intended to be limited to the particular forms disclosed. Rather, the invention is to cover all modifications, equivalents, and alternatives falling within the spirit and scope of the invention as defined by the appended claims.

### DETAILED DESCRIPTION OF THE INVENTION

In its most general aspect the present invention relates to a method for reversing an electrically driven piston rod in an injection device. By electrically driven is meant that the piston rod is driven by an electrical motor, such as a DC-motor. Optionally the shaft of the motor is operatively connected to the piston rod via some sort of gearing arrangement, such as a gear box. The motor performance is monitored by measuring the current provided to the motor and the position of the piston rod. By moving the piston rod in a forward direction a set dose of medicine can be injection from the injection device. When the piston rod has been moved forward a total distance which essentially equals the length of a medicine containing reservoir containing the medicine to be injected the piston rod needs to be reversed to its initial position so that the empty medicine containing reservoir can be replaced.

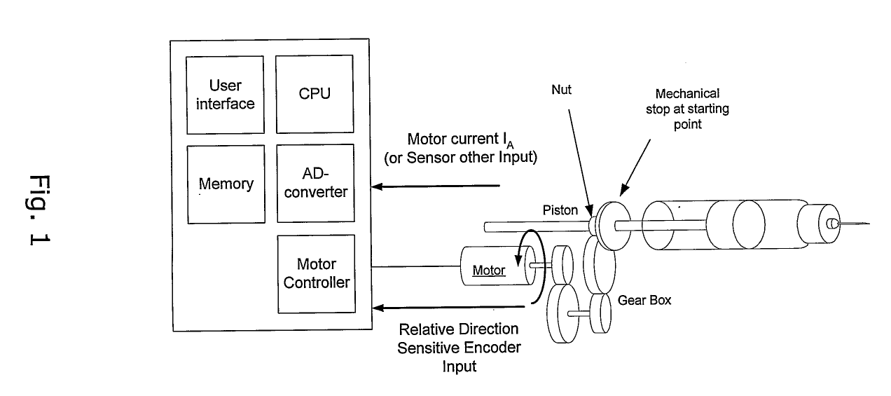

The control unit, motor, gear box, piston rod and medicine containing reservoir are shown in FIG. 1. The control unit comprises a user interface, a CPU, a memory module, A/D converter(s) for processing one or more incoming control signal(s), and a motor controller. In a preferred embodiment an A/D converter receives a control signal representing the provided current to the motor. The control signal representing the provided current is in analogue form, whereas the position signal can be in both digital and analogue form. The motor controller feeds the motor with an appropriate voltage level. Preferably, the motor controller comprises a H-bridge comprising four transistors of which two of the transistors cooperate to drive the motor in a forward direction, whereas two other transistors cooperate to drive the motor in a reverse direction. When driving the motor in the forward direction the supply voltage to the motor is varied by operating at least one of the two transistors in a pulse width modulation (PWM) mode. Similarly, the supply voltage can be varied using PWM when the motor is reversed to its original position.

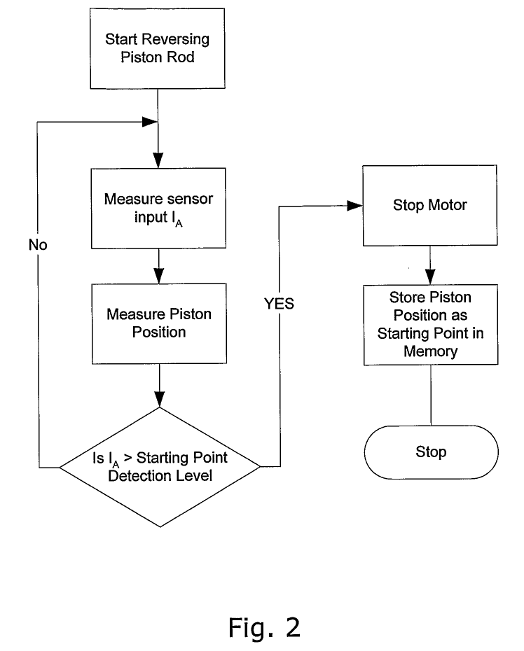

FIG. 2 shows a flow chart illustrating how the starting position of the piston rod determined and thereby how the system is calibrated. After having initiated the reversing procedure of the piston rod the current provided to the motor and the position of the piston rod is constantly measured. As long as the current provided to the motor stays below predetermined value the reversing of the piston rod is continued. However, if the current provided to the motor exceeds the predetermined value the motor is stopped and the position of the piston rod is determined and stored in the associated memory module. The stored position of the piston rod is used as the ideal position to be reached the next time the piston rod is to be reversed in order to replace a medicine containing medicine containing reservoir. Seen from a practical point of view the ideal position need not be reached exactly. Typically, a range corresponding to ±0.15 mm is defined around the ideal position meaning that as long as the piston rod is returned to a position falling within this range the piston rod is said to have been successfully returned to its stating position.

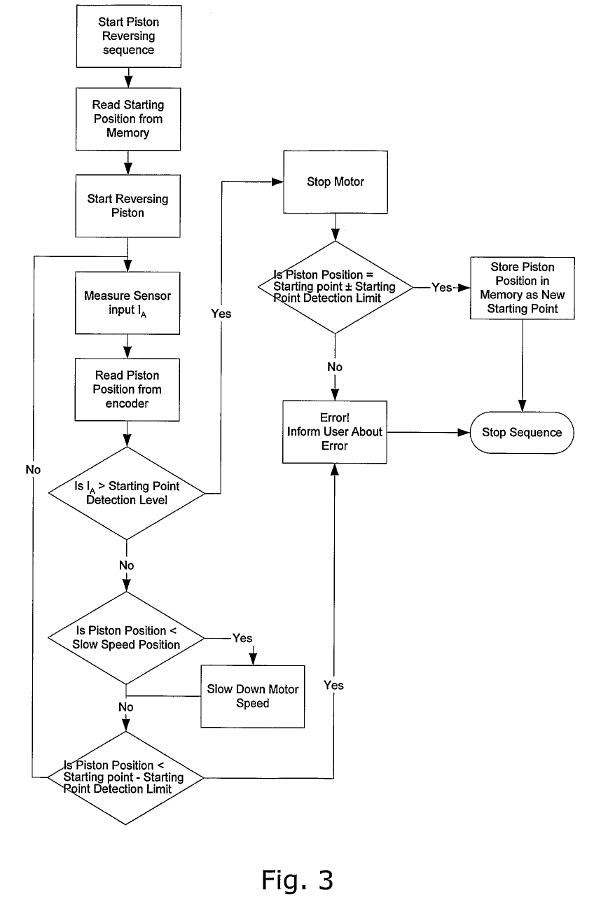

FIG. 3 shows a flow chart how the piston rod is reversed to its initial position. After the piston rod reversing sequence has been initiated an already stored starting position is read from the memory module of the control unit. When the starting position has been read the reversing of the piston rod is started by providing a supply voltage to the motor. The current provided to the motor and the position of the piston rod is constantly measured. If the level of the current provided to the motor exceeds a predetermined value, the motor is stopped and the position of the motor at its stopped position is compared to the starting position. If the position of the motor is within a predetermined range from the starting position (typically within a distance of approximate 0.15 mm) the control unit saves the actual position of the motor as a new starting position in the memory. This new starting position is used the next time the piston rod is to be reversed.

If the position of the motor falls outside the predetermined range an error signal is generated and the reversing sequence is stopped. Such situation could occur if the piston rod is in somehow prevented from reversing to its initial position—for example if the path to be followed by the piston rod is blocked.

If the level of the current provided to the motor stays below the predetermined level but the piston rod enters a range of predetermined positions the speed of the motor, and thereby the reversing speed of the piston rod, is decreased. The range of predetermined positions would typically be a region close to the starting position of the piston rod. In this way, the movement of the piston rod is slowed down before the starting position is reached. At this slow speed the current provided to the motor and the position of the piston rod is constantly measured. If the level of the current provided to the motor (running at a slower speed) exceeds a predetermined value, the motor is stopped and the position of the motor at its stopped position is compared to the starting position. If the position of the motor is within the predetermined range from the starting position the control unit saves the actual position of the motor as a new starting position in the memory. This new starting position is used the next time the piston rod is to be reversed. If the piston rod reverses to a position behind the starting position, i.e. if the position of the piston rod becomes smaller than the starting position minus half of the predetermined range, an error signal is generated informing the user of the injection device that the reversing procedure of the piston rod was not completed correctly. The injection device would most likely need to be repaired if this situation occurs.

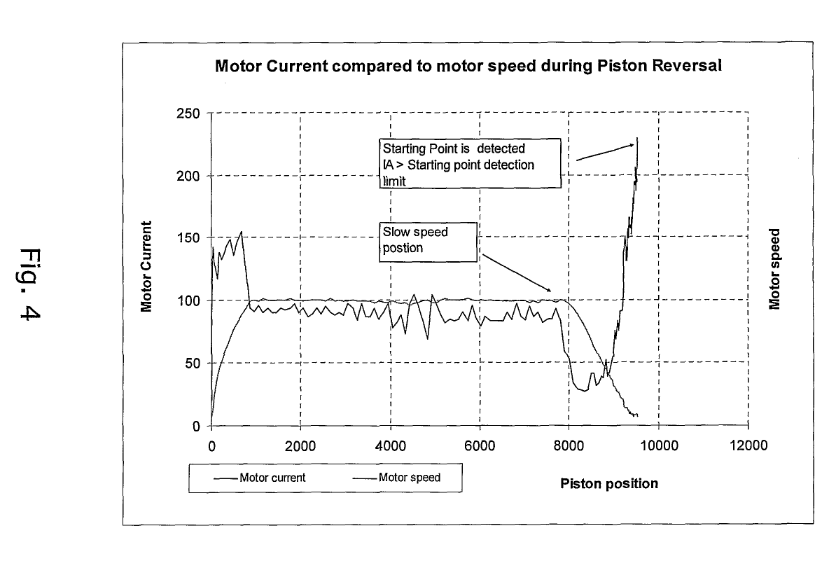

FIG. 4 shows the current provided to the motor vs. piston position and the motor speed vs. piston position during the reversing procedure. Looking at the motor speed it is seen that the motor is accelerated to its nominal speed which is kept constant until about 1.5 mm from the starting position. From this point the piston speed is linearly decreased. The current provided to the motor is higher than the nominal current as long as the motor speed is increased. When the nominal speed of the motor has been reached the current stabilises around the nominal level. When the piston rod enters the region where the motor is driven at a reduced speed the current drops until the piston rod enters a region near the piston rods starting position. In this region the piston rod becomes more difficult to move due to an increased mechanical friction between the piston rod and its surroundings. Due to this increased friction the current provided to the motor increases rapidly over a short distance and when the current reaches a predetermined level the motor is stopped and the position of the piston rod is determined and stored if it falls within a predetermined range.

1. A method for reversing a piston rod in an injection device, the piston rod being operatively connected to a motor shaft of an electrical motor so that a rotational movement of the motor shaft is transformed to a translational movement of the piston rod, the piston rod being adapted to be moved between two end positions, the method comprising:
providing a first supply voltage level to the electrical motor,
measuring sets of corresponding values of current provided to the motor and piston rod position,
shutting down the electrical motor in case the measured current provided to the motor exceeds a predetermined value, and
comparing a measured final position of the piston rod with a predetermined position range.

2. The method according to claim 1, wherein the position of the piston rod is measured by measuring a corresponding angular position of the motor shaft.

3. The method according to claim 1, further comprising generating an error signal if the measured final position of the piston rod is not included within the predetermined position range.

4. The method according to claim 1, further comprising generating an approving signal if the measured final position of the piston rod is included within the predetermined position range.

5. The method according to claim 4, further comprising storing the final position of the piston rod in an associated memory module for later use.

6. A method for reversing a piston rod in an injection device, the piston rod being operatively connected to a motor shaft of an electrical motor so that a rotational movement of the motor shaft is transformed to a translational movement of the piston rod, the piston rod being adapted to be moved between two end positions, the method comprising:
providing a first supply voltage level to the electrical motor,
measuring sets of corresponding values of current provided to the motor and piston rod position, and
providing a second supply voltage level to the electrical motor in case the measured position of the piston rod equals predetermined positions, the second supply voltage level having a RMS value being smaller than the RMS value of the first supply voltage level.

7. The method according to claim 6, wherein the position of the piston rod is measured by measuring a corresponding angular position of the motor shaft.

8. The method according to claim 6, further comprising shutting down the electrical motor in case the current provided to the motor exceeds a predetermined value.

9. The method according to claim 8, further comprising comparing a measured final position of the piston rod with a predetermined position range.

10. The method according to claim 9, further comprising generating an error signal if the measured final position is not included within the predetermined position range.

11. The method according to claim 9, further comprising generating an approving signal if the measured final position is included within the predetermined position range.

12. The method according to claim 11, further comprising storing the final position of the piston rod in an associated memory module for later use.

13. An apparatus for reversing a piston rod in an injection device, the piston rod being operatively connected to a motor shaft of an electrical motor so that a rotational movement of the motor shaft is transformed to a translational movement of the piston rod, the piston rod being adapted to be moved between two end positions, the apparatus comprising:
means for providing a first supply voltage level to the electrical motor,
means for measuring sets of corresponding values of current provided to the motor and piston rod position, and
means for shutting down the electrical motor in case the measured current provided to the motor exceeds a predetermined value.

14. An apparatus for reversing a piston rod in an injection device, the piston rod being operatively connected to a motor shaft of an electrical motor so that a rotational movement of the motor shaft is transformed to a translational movement of the piston rod, the piston rod being adapted to be moved between two end positions, the apparatus comprising:
means for providing a first supply voltage level to the electrical motor,
means for measuring sets of corresponding values of current provided to the motor and piston rod position, and
means for providing a second supply voltage level to the electrical motor if the measured position of the piston rod equals predetermined positions, the second supply voltage level having a RMS value being smaller than the RMS value of the first supply voltage level.
